(19) United States
(12) Patent Application Publication
(10) Pub. No.: US 2016/0331986 A1
(43) Pub. Date: Nov. 17, 2016

(54) WEARABLE CARDIAC DEFIBRILLATOR SYSTEM WITH COMMUNICATING BATTERY CHARGER

(71) Applicant: West Affum Holdings Corp., Grand Cayman (KY)

(72) Inventors: Daniel R. Piha, Bellevue, WA (US); Gregory T. Kavounas, Bellevue, WA (US)

(21) Appl. No.: 14/727,192

(22) Filed: Jun. 1, 2015

Related U.S. Application Data
(60) Provisional application No. 62/159,901, filed on May 11, 2015.

(51) Int. Cl. A61N 1/39; A61N 1/04; G08B 21/18

(52) U.S. Cl. CPC A61N 1/3975; G08B 21/18; A61N 1/3968; A61N 1/3993; A61N 1/046; A61N 1/3925

(57) ABSTRACT

A WCD system includes a support structure configured to be worn by a patient, a portable antenna coupled to the support structure, a rechargeable WCD battery, and a charger for recharging the WCD battery. The portable antenna is configured to transmit wirelessly to the charger an encoded message from components that are worn by the patient on the support structure. The charger also includes a charger antenna configured to receive the encoded message, and a user interface configured to output a human-perceptible indication responsive to the received encoded message. Accordingly, a message communicated from the WCD system will appear at the location of the charger, which is a more predictable location for a human attendant than the location of the patient.

### CROSS REFERENCE TO RELATED PATENT APPLICATIONS

This patent application claims priority from U.S. Provisional Patent Application Ser. No. 62/159,901 filed on May 11, 2015, the disclosure of which is hereby incorporated by reference.

### BACKGROUND

When people suffer from some types of heart arrhythmias, the result may be that blood flow to various parts of the body is reduced. Some arrhythmias may even result in a Sudden Cardiac Arrest (SCA). SCA can lead to death very quickly, e.g. within 10 minutes, unless treated in the interim.

Some people have an increased risk of SCA. People at a higher risk include individuals who have had a heart attack, or a prior SCA episode. A frequent recommendation is for these people to receive an Implantable Cardioverter Defibrillator (“ICD”). The ICD is surgically implanted in the chest, and continuously monitors the person's electrocardiogram (“ECG”). If certain types of heart arrhythmias are detected, then the ICD delivers an electric shock through the heart.

After being identified as having an increased risk of an SCA, and before receiving an ICD, these people are sometimes given a wearable cardiac defibrillator (“WCD”) system. A WCD system typically includes a harness, vest, or other garment that the patient is to wear. The WCD system includes a defibrillator and electrodes, coupled to the harness, vest, or other garment. When the patient wears the WCD system, the external electrodes may then make good electrical contact with the patient's skin, and therefore can help determine the patient's ECG. If a shockable heart arrhythmia is detected, then the defibrillator delivers the appropriate electric shock through the patient's body, and thus through the heart.

### BRIEF SUMMARY

The present description gives instances of wearable cardiac defibrillator (WCD) systems and methods, the use of which may help overcome problems and limitations of the prior art.

In embodiments, a WCD system includes a support structure configured to be worn by a patient, a portable antenna coupled to the support structure, a rechargeable WCD battery, and a charger for recharging the WCD battery. The portable antenna is configured to transmit wirelessly to the charger an encoded message from components that are worn by the patient on the support structure. The charger also includes a charger antenna configured to receive the encoded message, and a user interface configured to output a human-perceptible indication responsive to the received encoded message.

An advantage is that status messages and alerts communicated from the WCD system will appear at the location of the charger, which is a more predictable location for a human attendant than the location of the patient. Accordingly, an attendant at a facility, a family member, or a coworker can be more certain of receiving the message by being near the charger, without having to follow the patient everywhere at all times. Moreover, such status messages and alerts may be provided while, for example, the patient is in the comfort of their own home, and even sleeping, and may increase the probability of intervention before inappropriately delivered therapy.

In embodiments, a WCD system includes a support structure configured to be worn by a patient, a portable antenna and a speaker coupled to the support structure, a rechargeable WCD battery, and a charger for recharging the WCD battery. The charger also includes a siren actuator that can receive a siren input by an attendant, and a charger antenna that can transmit wirelessly a siren command responsive to the received siren input. The portable antenna on the support structure may receive the siren command and, as a result, the speaker can emit a sound. This way the attendant can communicate a message to the patient, or increase the sound so as to locate the patient.

In embodiments, a WCD system includes a support structure configured to be worn by a patient who may use a mobile communication device, a rechargeable WCD battery, and a combination charger for recharging the WCD battery and the mobile communication device.

These and other features and advantages of this description will become more readily apparent from the Detailed Description, which proceeds with reference to the associated drawings in which:

### BRIEF DESCRIPTION OF THE DRAWINGS

FIG. 1 is a diagram of components of a sample wearable cardiac defibrillator (WCD) system, made according to embodiments.

FIG. 2 is a diagram showing sample components of an external defibrillator, such as the one belonging in the WCD system of FIG. 1, and which is made according to embodiments.

FIG. 3 is a diagram of sample components of a WCD system that is made according to embodiments.

FIG. 4 is a diagram of a speaker that could be included in the User Interface of a charger of a WCD system made according to embodiments.

FIG. 5 is a diagram of a screen that could be included in the User Interface of a charger of a WCD system made according to embodiments.

FIG. 6 is a diagram of a sample panel that could be included in the User Interface of a charger of a WCD system made according to embodiments.

FIG. 7 is a flowchart for illustrating methods according to embodiments.

FIG. 8 is a diagram of components of a sample wearable cardiac defibrillator (WCD) system, made according to embodiments.

FIG. 9 is a flowchart for illustrating methods according to embodiments.

FIG. 10 is a diagram of components of a sample wearable cardiac defibrillator (WCD) system, made according to embodiments.

FIG. 11 is a top view of a combination charger of a WCD system, which is made according to embodiments.

### DETAILED DESCRIPTION

As has been mentioned, the present description is about WCD systems and methods. Embodiments are now described in more detail.

A wearable cardiac defibrillator (WCD) system made according to embodiments has a number of components. These components can be provided separately as modules that can be interconnected, or can be combined with other components, etc.

A component of a WCD system can be a support structure, which is configured to be worn by the patient. The support structure can be any structure suitable for wearing, such as a harness, a vest, a half-vest—for example over the left side of the torso that positions electrodes on opposite sides of the heart, one or more belts that are configured to be worn horizontally or possibly vertically over a shoulder, another garment, and so on. The support structure can be implemented in a single component or multiple components. For example, a support structure may have a top component resting on the shoulders, for ensuring that the defibrillation electrodes will be in the appropriate positions for defibrillating, and a bottom component resting on the hips, for carrying the bulk of the weight of the defibrillator. A single component embodiment could be with a belt around at least the torso. Other embodiments could use an adhesive structure or another way for attaching to the patient, without encircling any part of the body. There can be other examples.

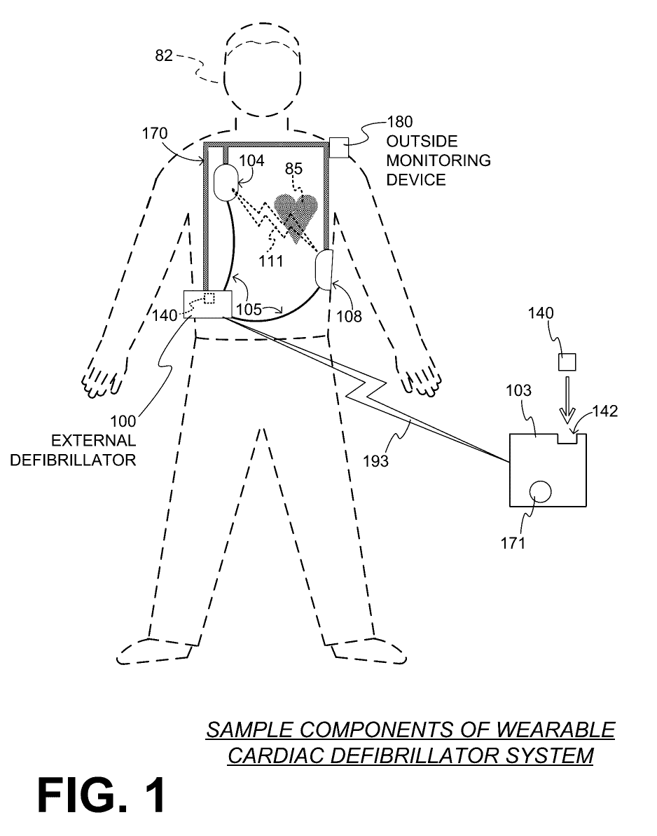

FIG. 1 depicts components of a WCD system made according to embodiments, as it might be worn by a person 82. A person such as person 82 may also be referred to as a patient and/or wearer, since that person wears components of the WCD system.

In FIG. 1, a generic support structure 170 is shown relative to the body of patient 82, and thus also relative to his or her heart 85. Structure 170 could be a harness, a vest, a half-vest, one or more belts, or a garment, etc., as per the above. Structure 170 could be implemented in a single component, or multiple components, and so on. Structure 170 is wearable by patient 82, but the manner of wearing it is not depicted, as structure 170 is depicted only generically in FIG. 1.

A wearable cardiac defibrillator (WCD) system is configured to defibrillate a patient who is wearing it, by delivering electrical charge to the patient's body in the form of an electric shock delivered in one or more pulses. FIG. 1 shows a sample external defibrillator 100, and sample defibrillation electrodes 104, 108, which are coupled to external defibrillator 100 via electrode leads 105. Defibrillator 100 and defibrillation electrodes 104, 108 are coupled to support structure 170. As such, many of the components of defibrillator 100 can be therefore coupled to support structure 170. When defibrillation electrodes 104, 108 make good electrical contact with the body of patient 82, defibrillator 100 can administer, via electrodes 104, 108, a brief, strong electric pulse 111 through the body. Pulse 111, also known as a defibrillation shock or therapy shock, is intended to go through and restart heart 85, in an effort to save the life of patient 82. Pulse 111 can further include one or more pacing pulses, and so on.

A prior art defibrillator typically decides whether to defibrillate or not based on an electrocardiogram (“ECG”) signal of the patient. However, defibrillator 100 can defibrillate, or not defibrillate, also based on other inputs.

The WCD system may optionally include an outside monitoring device 180. Device 180 is called an “outside” device because it is provided as a standalone device, for example not within the housing of defibrillator 100. Device 180 can be configured to monitor at least one local parameter. A local parameter can be a parameter of patient 82, or a parameter of the WCD system, or a parameter of the environment, as will be described later in this document.

Optionally, device 180 is physically coupled to support structure 170. In addition, device 180 can be communicatively coupled with other components, which are coupled to support structure 170. Such communication can be implemented by a communication module, as will be deemed applicable by a person skilled in the art in view of this disclosure.

Defibrillator 100 may include a WCD battery 140, as will be described later in more detail. In FIG. 1, WCD battery 140 is shown in two places—both within defibrillator 100 for operation and removed from defibrillator 100 so that it can be charged.

A WCD system according to embodiments may also include a charger 103 for charging WCD battery 140. Charger 103 may operate conductively, i.e. via one or more pins making electrical contact, or inductively, i.e. via communicating coils. Charger 103 may have a battery receptacle 142. Charger 103 may also have a user interface 171, and a charger antenna (not shown in FIG. 1). The charger antenna may establish a communication link (“comlink”) 193 with a portable antenna of defibrillator 100 (not shown in FIG. 1). Charger 103 can be made with appropriate components for these functions, and so on.

These components are now described in more detail.

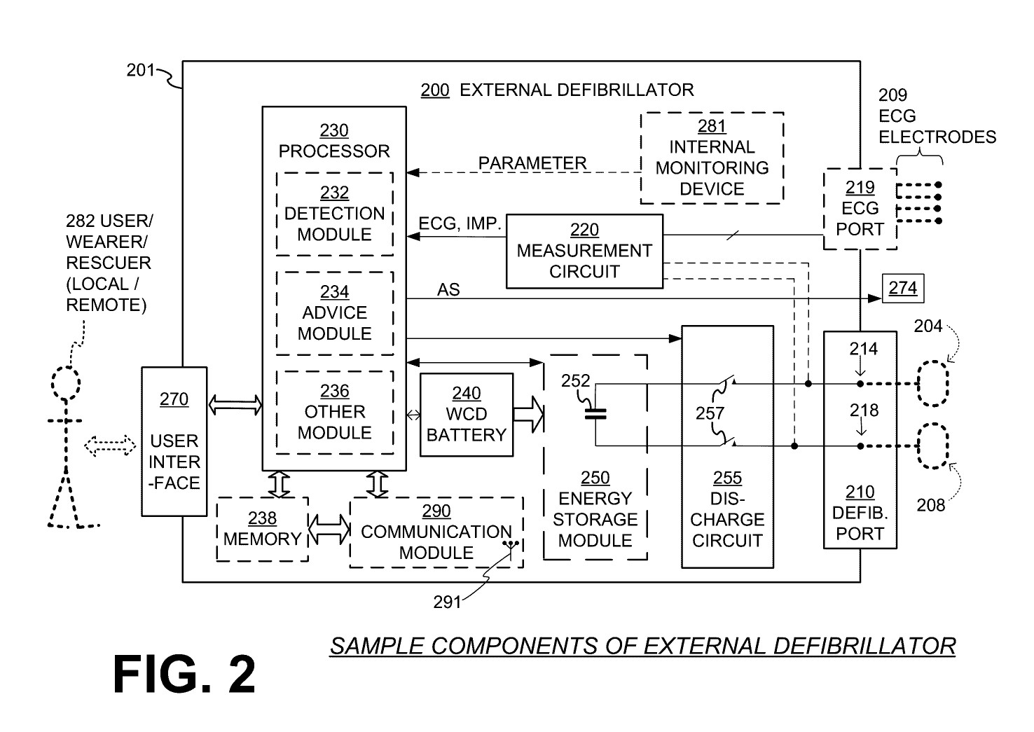

FIG. 2 is a diagram showing components of an external defibrillator 200, made according to embodiments. These components can be, for example, included in external defibrillator 100 of FIG. 1. The components shown in FIG. 2 can be provided in a housing 201, which is also known as casing 201.

External defibrillator 200 is intended for a patient who would be wearing it, such as patient 82 of FIG. 1. Defibrillator 200 may further include a user interface 270 for a user 282. User 282 can be patient 82, also known as wearer 82. Or user 282 can be a local rescuer at the scene, such as a bystander who might offer assistance, or a trained person. Or, user 282 might be a remotely located trained caregiver in communication with the WCD system.

User interface 270 can be made in any number of ways. User interface 270 may include output devices, which can be visual, audible or tactile, for communicating to a user. For example, an output device can be a light, or a screen to display what is detected and measured, and provide visual feedback to rescuer 282 for their resuscitation attempts, and so on. Another output device can be a speaker, which can be configured to issue voice prompts, etc. Sounds, images, vibrations, and anything that can be perceived by user 282 can also be called human perceptible indications. User interface 270 may also include input devices for receiving inputs from users. Such input devices may additionally include various controls, such as pushbuttons, keyboards, touchscreens, a microphone, and so on. An input device can be a cancel switch, which is sometimes called a “live-man” switch, or an “I am alive” switch. In some embodiments, actuating the cancel switch is an intervention by a human that can prevent the impending delivery of a shock that would be advised automatically inappropriately by the WCD system.

Defibrillator 200 may include an internal monitoring device 281. Device 281 is called an “internal” device because it is incorporated within housing 201. Monitoring device 281 can monitor patient parameters, patient physiological parameters, system parameters and/or environmental parameters, all of which can be called patient data. In other words, internal monitoring device 281 can be complementary or an alternative to outside monitoring device 180 of FIG. 1. Allocating which of the system parameters are to be monitored by which monitoring device can be done according to design considerations.

Patient physiological parameters include, for example, those physiological parameters that can be of any help in detecting by the wearable defibrillation system whether the patient is in need of a shock, plus optionally their medical history and/or event history. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring device could include a perfusion sensor, a pulse oximeter, a Doppler device for detecting blood flow, a cuff for detecting blood pressure, an optical sensor, illumination detectors and perhaps sources for detecting color change in tissue, a motion sensor, a device that can detect heart wall movement, a sound sensor, a device with a microphone, an SpO2 sensor, and so on. Pulse detection is taught at least in Physio-Control's U.S. Pat. No. 8,135,462, which is hereby incorporated by reference in its entirety. In addition, a person skilled in the art may implement other ways of performing pulse detection.

In some embodiments, the local parameter is a trend that can be detected in a monitored physiological parameter of patient 82. A trend can be detected by comparing values of parameters at different times. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2 or CO2; f) respiratory function, respiratory rate, etc.; g) motion, level of activity; and so on. Once a trend is detected, it can be stored and/or reported via a communication link, along perhaps with a warning. From the report, a physician monitoring the progress of patient 82 will know about a condition that is either not improving or deteriorating.

Patient state parameters include recorded aspects of patient 82, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A wearable cardiac defibrillator (WCD) system made according to embodiments may include a motion detector. In embodiments, a motion detector can be implemented within monitoring device 180 or monitoring device 281. Such a motion detector can be configured to detect a motion event. In response, the motion detector may render or generate from the detected motion event a motion detection input that can be received by a subsequent device or functionality. A motion event can be defined as is convenient, for example a change in motion from a baseline motion or rest, etc. Such a motion detector can be made in many ways as is known in the art, for example by using an accelerometer.

System parameters of a WCD system can include system identification, battery status, system date and time, reports of self-testing, records of data entered, records of episodes and intervention, and so on.

Environmental parameters can include ambient temperature and pressure. A humidity sensor may provide information as to whether it is likely raining. Presumed patient location could also be considered an environmental parameter. The patient location could be presumed if monitoring device 180 or 281 includes a GPS location sensor as per the above.

Defibrillator 200 typically includes a defibrillation port 210, such as a socket in housing 201. Defibrillation port 210 includes electrical nodes 214, 218. Leads of defibrillation electrodes 204, 208, such as leads 105 of FIG. 1, can be plugged into defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer the electrical charge that has been stored in energy storage module 250. The electric charge will be the shock for defibrillation, pacing, and so on.

Defibrillator 200 may optionally also have an ECG port 219 in housing 201, for plugging in sensing electrodes 209, which are also known as ECG electrodes and ECG leads. It is also possible that sensing electrodes 209 can be connected continuously to ECG port 219, instead. Sensing electrodes 209 can help sense an ECG signal, e.g. a 12-lead signal, or a signal from a different number of leads, especially if they make good electrical contact with the body of the patient. Sensing electrodes 209 can be attached to the inside of support structure 170 for making good electrical contact with the patient, similarly as defibrillation electrodes 204, 208.

Optionally a WCD system according to embodiments also includes a fluid that it can deploy automatically between the electrodes and the patient skin. The fluid can be conductive, such as by including an electrolyte, for making a better electrical contact between the electrode and the skin. Electrically speaking, when the fluid is deployed, the electrical impedance between the electrode and the skin is reduced. Mechanically speaking, the fluid may be in the form of a low-viscosity gel, so that it does not flow away, after it has been deployed. The fluid can be used for both defibrillation electrodes 204, 208, and sensing electrodes 209.

The fluid may be initially stored in a fluid reservoir, not shown in FIG. 2, which can be coupled to the support structure. In addition, a WCD system according to embodiments further includes a fluid deploying mechanism 274. Fluid deploying mechanism 274 can be configured to cause at least some of the fluid to be released from the reservoir, and be deployed near one or both of the patient locations, to which the electrodes are configured to be attached to the patient. In some embodiments, fluid deploying mechanism 274 is activated responsive to receiving activation signal AS from processor 230, prior to the electrical discharge.

Defibrillator 200 also includes a measurement circuit 220. Measurement circuit 220 receives physiological signals of the patient from ECG port 219, if provided. Even if defibrillator 200 lacks ECG port 219, measurement circuit 220 can obtain physiological signals through nodes 214, 218 instead, when defibrillation electrodes 204, 208 are attached to the patient. In these cases, the patient's ECG signal can be sensed as a voltage difference between electrodes 204, 208. Plus, impedance between electrodes 204, 208 and/or the connections of ECG port 219 can be sensed. Sensing the impedance can be useful for detecting, among other things, whether these electrodes 204, 208 and/or sensing electrodes 209 are not making good electrical contact with the patient's body. These patient physiological signals can be sensed, when available. Measurement circuit 220 can then render or generate information about them as physiological inputs, data, other signals, etc. More strictly speaking, the information rendered by measurement circuit 220 is output from it, but this information can be called an input because it is received by a subsequent device or functionality as an input.

Defibrillator 200 also includes a processor 230. Processor 230 may be implemented in any number of ways. Such ways include, by way of example and not of limitation, digital and/or analog processors such as microprocessors and Digital Signal Processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 can be considered to have a number of modules. One such module can be a detection module 232. Detection module 232 can include a Ventricular Fibrillation (“VF”) detector. The patient's sensed ECG from measurement circuit 220, which can be available as physiological inputs, data, or other signals, may be used by the VF detector to determine whether the patient is experiencing VF. Detecting VF is useful, because VF results in SCA. Detection module 232 can also include a Ventricular Tachycardia (“VT”) detector, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more of ECG signals that are captured according to embodiments, and determining whether a shock criterion is met. The determination can be made from a rhythm analysis of the captured ECG signal or otherwise. Accordingly, processor 230 can be configured to determine that electrical charge stored in the defibrillator should be discharged through the patient, sometimes if there is no timely intervention by a human.

In some embodiments, when the decision is to shock, an electrical charge is delivered to the patient. Delivering the electrical charge is also known as discharging. Shocking can be for defibrillation, pacing, and so on.

Processor 230 can include additional modules, such as other module 236, for other functions. In addition, if internal monitoring device 281 is indeed provided, it may be operated in part by processor 230, etc.

Defibrillator 200 optionally further includes a memory 238, which can work together with processor 230. Memory 238 may be implemented in any number of ways. Such ways include, by way of example and not of limitation, volatile memories, nonvolatile memories (NVM), read-only memories (ROM), random access memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read and execute. More particularly, the programs can include sets of instructions in the form of code, which processor 230 may be able to execute upon reading. Executing is performed by physical manipulations of physical quantities, and may result in functions, processes, actions and/or methods to be performed, and/or the processor to cause other devices or components or blocks to perform such functions, processes, actions and/or methods. The programs can be operational for the inherent needs of processor 230, and can also include protocols and ways that decisions can be made by advice module 234. In addition, memory 238 can store prompts for user 282, if this user is a local rescuer. Moreover, memory 238 can store data. The data can include patient data, system data and environmental data, for example as learned by internal monitoring device 281 and outside monitoring device 180. The data can be stored in memory 238 before it is transmitted out of defibrillator 200, or stored there after it is received by defibrillator 200.

Defibrillator 200 may also include a WCD battery 240 that can be configured to store an electrical charge, so as to serve as a power source for the WCD system. WCD battery 140 can be as WCD battery 240. In some embodiments, WCD battery 240 is controlled by processor 230. WCD battery 240 can be rechargeable, so as to enable portability of defibrillator 200. In embodiments, therefore, WCD battery 240 can be physically removed from, and inserted back into, defibrillator 200.

Defibrillator 200 moreover includes a discharge circuit 255, which therefore is coupled to the support structure (not shown in FIG. 2). When the decision is to shock, processor 230 can be configured to control discharge circuit 255 to discharge through the patient the electrical charge stored in WCD battery 240. When so controlled, circuit 255 can permit this stored energy to be discharged to nodes 214, 218, and from there also to defibrillation electrodes 204, 208. Circuit 255 can include one or more switches 257. Switches 257 can be made in a number of ways, such as by an H-bridge, and so on. Circuit 255 can also be controlled via user interface 270.

In some embodiments, defibrillator 200 additionally includes an energy storage module 250, which can thus be coupled to the support structure of the WCD system. Energy storage module 250 can be configured to receive the electrical charge stored in WCD battery 240, and to store the electrical charge it receives this way. In such embodiments, discharge circuit 255 is configured to discharge the electrical charge that becomes stored in energy storage module 250. In typical implementations, module 250 includes a capacitor 252, which can be a single capacitor or a system of capacitors, and so on. As described above, capacitor 252 can store the energy in the form of electrical charge, for delivering to the patient.

WCD battery 240 can be further configured to be electrically coupled to, and uncoupled from, discharge circuit 255. More particularly, this can take place when WCD battery 240 is physically inserted into, and removed from, defibrillator 200, respectively.

Defibrillator 200 can optionally include a communication module 290, for establishing one or more wired or wireless communication links with other devices of other entities, such as a remote assistance center, Emergency Medical Services (EMS), and so on. Module 290 may also include an antenna 291, portions of a processor, and other sub-components as may be deemed necessary by a person skilled in the art. This way, data and commands can be communicated, such as patient data, event information, therapy attempted, CPR performance, system data, environmental data, notifications such as for impending shock and option to intervene, and so on. Antenna 291 can be called a portable antenna because it is ultimately coupled to the support structure, and therefore is one of the components of the WCD system that move with the patient, and not one of the stationary components such as charger 103 that is typically plugged into an electrical wall outlet.

Defibrillator 200 can optionally include other components.

Returning to FIG. 1, in embodiments, one or more of the components of the shown WCD system have been customized for patient 82. This customization may include a number of aspects. For instance, support structure 170 can be fitted to the body of patient 82. For another instance, baseline physiological parameters of patient 82 can be measured, such as the heart rate of patient 82 while resting, while walking, motion detector outputs while walking, etc. Such baseline physiological parameters can be used to customize the WCD system, in order to make its diagnoses more accurate, since bodies behave differently. For example, such parameters can be stored in a memory of the WCD system, and so on.

A programming interface can be made according to embodiments, which receives such measured baseline physiological parameters. Such a programming interface may input automatically in the WCD system the baseline physiological parameters, along with other data.

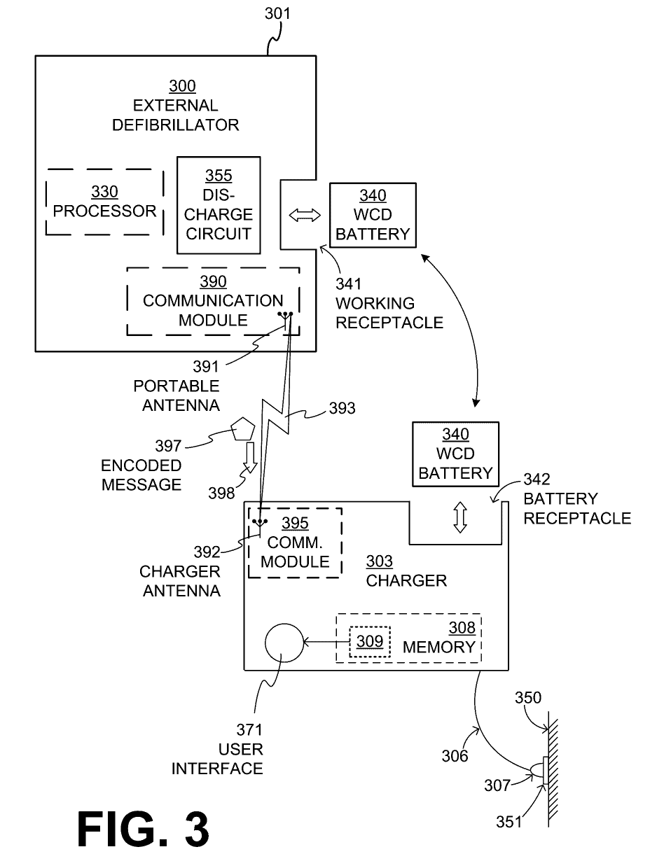

FIG. 3 is a diagram of sample components of a WCD system that is made according to embodiments. The WCD system includes an external defibrillator 300 that is provided in a housing 301. External defibrillator 300 can be as external defibrillator 200 or 100, and can be coupled to a support structure such as support structure 170. The WCD system moreover includes components that are coupled to a support structure that is not shown in FIG. 3, and could be as support structure 170. In the example of FIG. 3, many of these components are part of external defibrillator 300. More particularly, external defibrillator 300 includes a discharge circuit 355 that can be as discharge circuit 255, a portable antenna 391 that can be as portable antenna 291, and optionally a processor 330 that can be as processor 230. Portable antenna 391 can be configured to transmit wirelessly an encoded message 397. Optionally portable antenna 291 is part of a communication module 390 that can be as communication module 290.

The WCD system also includes a rechargeable WCD battery 340, which is shown in two places. WCD battery 340 can be inserted in a working receptacle 341 for powering external defibrillator 300. WCD battery 340 can also be removed from working receptacle 341, for being recharged by a charger 303 that is made according to embodiments. In some embodiments, another WCD battery is provided (not shown), so that one battery can be in external defibrillator 300 while the other is being recharged, and so on.

In some embodiments, charger 303 is as charger 103. In embodiments, charger 303 can be configured to be coupled to an electrical wall outlet 351 of a wall 350, so as to receive electrical power. More particularly, charger 303 may have an electrical cord 306 with a plug 307 that can be plugged into electrical wall outlet 351.

Charger 303 can be configured to charge WCD battery 340 from the electrical power received via electrical cord 306, while WCD battery 340 is uncoupled from external defibrillator 300 and is thus also uncoupled from discharge circuit 355. This charging process, therefore, causes WCD battery 340 to store an electrical charge, which can be used as mentioned above. Preferably, charger 303 includes a battery receptacle 342 that is configured to receive WCD battery 340 while charger 303 charges WCD battery 340.

Charger 303 may also include a charger antenna 392 that is configured to receive encoded message 397 from portable antenna 391. More particularly, charger 303 may have its own communication module 395 that is powered by the electrical power. Charger antenna 392 can be part of communication module 395. Encoded message 397 may travel via a comlink 393 along a direction 398 and thus be received by charger antenna 392, which is configured to receive it. Comlink 393 may be implemented via a Bluetooth Smart (Bluetooth Low Energy) connection, or other equivalent or suitable technology.

Charger 303 may further include a user interface 371. User interface 371 can be configured to output a human-perceptible indication, responsive to charger antenna 392 receiving encoded message 397. The human-perceptible indication may be auditory, visual, etc., and may be output regardless of whether WCD battery 340 is being charged or not.

As will be seen later in this document, the human-perceptible indication often includes a message or notification to a person such as the patient or an attendant. In some embodiments, only a short code need be transmitted for a full message to be played out as the human-perceptible indication. Examples are now described.

In some embodiments, charger 303 further includes a memory 308. Memory 308 can be configured to store a data group 309. Data group 309 can be one or more data files or other data structure that encodes the message that is to be played out from user interface 371 as the human-perceptible indication. In such embodiments, data group 309 can be sent to user interface 371, responsive to charger antenna 392 receiving encoded message 397. Accordingly, the human-perceptible indication can be output by user interface 371 responsive to data group 309 being sent to user interface 371.

In some of these embodiments, memory 308 is configured to store a plurality of data groups, i.e. more than one memory groups so as to make it possible to communicate more than one message. A message code may be decoded from the encoded message 397, and at least a certain one of the data groups can be selected responsive to the message code. The selected certain data group can be sent to user interface 371, and so on.

The human-perceptible indication can include a number of messages. For example, the message can be about a system parameter of the WCD system, or about a value of a monitored parameter of the patient, or a trend, or an alarm, or about treating the patient, etc.

In some embodiments, processor 330 is configured to determine that the stored electrical charge should be discharged through the patient. This can be implemented, for example, by advice module 234. In such embodiments, the human-perceptible indication can include a message about the determination. Encoded message 397 may be sent prior to the discharge, include the warning to stand clear, and so on.

In some of these embodiments, processor 330 can be configured to determine that the stored electrical charge should be discharged through the patient at a certain moment, unless there is a timely intervention by a human. In such embodiments, the human-perceptible indication can include a message about the determination, and encoded message 397 can be transmitted at least 4 sec or longer prior to the certain moment. This time would give a human an opportunity to intervene, for example by pressing the “I am alive” switch. The message could further instruct as to how to perform the intervention.

User interface 371 can thus output secondary human perceptible indications so as to attract attention also from an attendant, who may be in the vicinity of charger 371. This can be especially useful for diminishing the risk that a distracted or asleep patient will not hear the warning for an impending shock and thus be subjected to it. Different examples are now described for user interface 371 and the human-perceptible indications it can output.

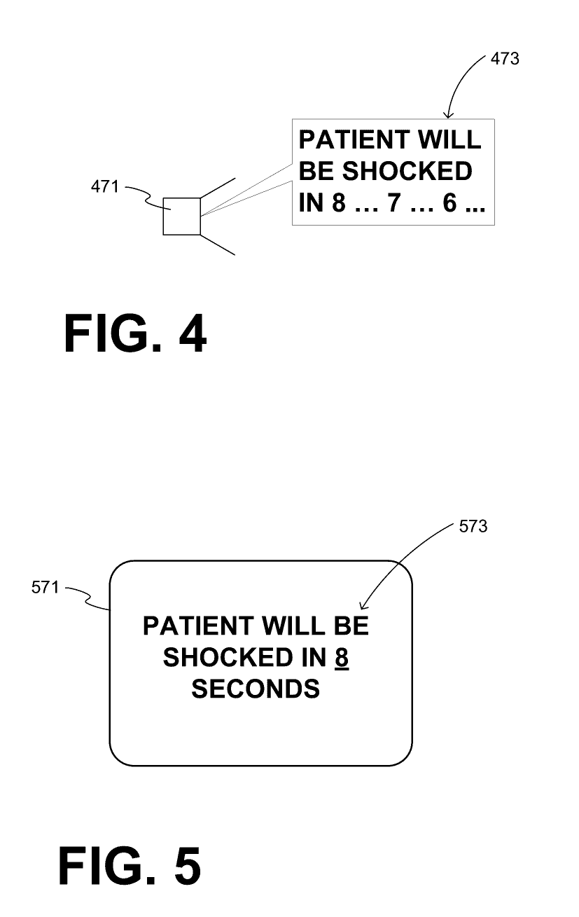

As can be seen in FIG. 4, the human-perceptible indication includes a sound 473, and the user interface includes a speaker 471 configured to play sound 473. Speaker 471 can be powerful, so that sound 473 can be played loudly. The user interface may include a speaker driver, the data group can be a sound file, etc.

As can be seen in FIG. 5, the human-perceptible indication includes a visual message 573, and the user interface includes a screen 571 configured to display visual message 573. The user interface may include a display driver, the data group can be a image file, etc.

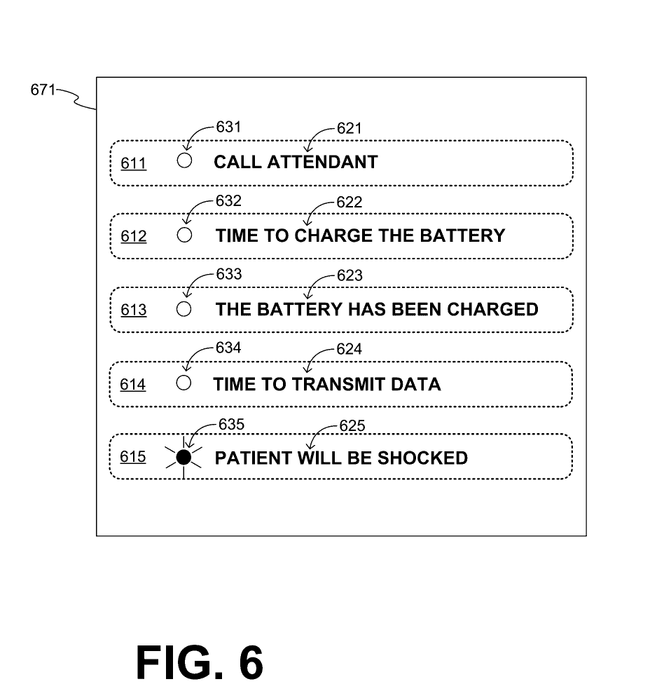

As can be seen in FIG. 6, the human-perceptible indication can include one or more visual messages 621, 622, 623, 624, 625. The user interface may include a panel 671 with one or more portions 611, 612, 613, 614, 615 that respectively display visual messages 621, 622, 623, 624, 625.

The user interface may further include one or more light sources 631, 632, 633, 634, 635 that are associated with the respective panel portions 611, 612, 613, 614, 615. Association can be implemented as seen in FIG. 6, namely by placing light sources 631, 632, 633, 634, 635 next to visual messages 621, 622, 623, 624, 625. In this example, light source 635 is illuminated, which draws attention to message 625. Alternately, association can be implemented by making panel portions 611, 612, 613, 614, 615 translucent, and placing light sources 631, 632, 633, 634, 635 behind panel portions 611, 612, 613, 614, 615. Light sources 631, 632, 633, 634, 635 can be bright, flashing, etc., so as to attract attention.

Moreover, embodiments shown in FIG. 3 may be extended by features and embodiments shown later in this document.

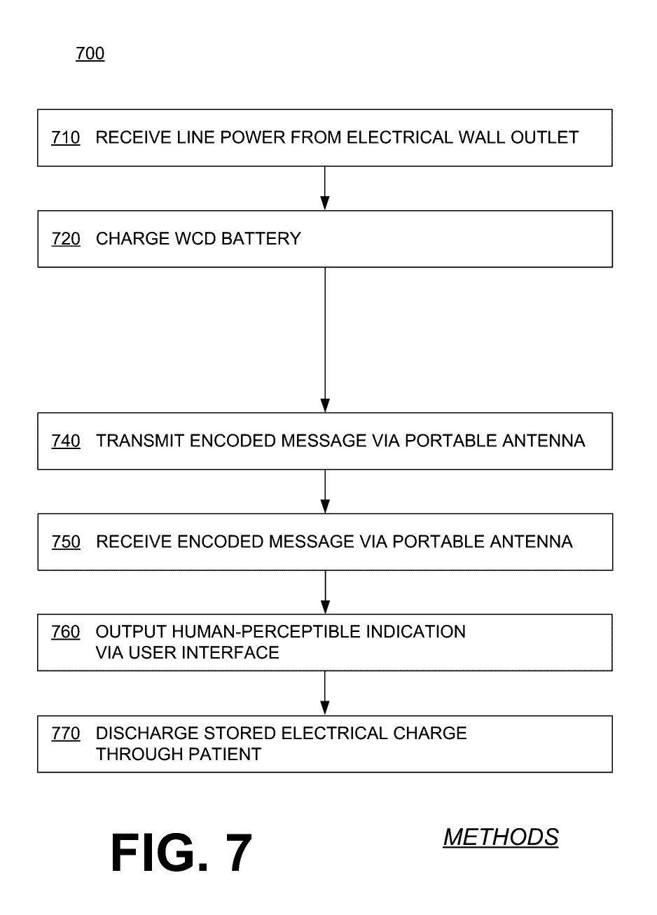

FIG. 7 shows a flowchart 700 for describing methods according to embodiments. The methods of flowchart 700 may be practiced by embodiments described above.

According to an operation 710, electrical power is received in the charger. The electrical power may be received from an electrical wall outlet.

According to another operation 720, a WCD battery may be charged with the electrical power received at operation 710. This charging will result in the WCD battery to store an electrical charge. In some embodiments, the charger further includes a communicator receptacle configured to receive a mobile communication device that has a suitable rechargeable battery that is also known as a communicator battery. In such embodiments, the communicator battery can be further charged from the electrical power, while the mobile communication device has been received in the communicator receptacle.

According to another operation 740, an encoded message can be transmitted wirelessly via the portable antenna. The encoded message can be transmitted when there is a message to be communicated.

According to another operation 750, the encoded message may be received via the portable antenna.

According to another operation 760, a human-perceptible indication can be output via the user interface. This may happen responsive to operation 750. In some embodiments, the charger further includes a memory, and one or more data groups are stored in the memory. In some of these embodiments, a message code is decoded from the received encoded message, and at least a certain one of the data groups is selected responsive to the message code. The selected data group can be sent to the user interface responsive to operation 750, and the human-perceptible indication can be output responsive to the data group being sent to the user interface.

According to another operation 770, the stored electrical charge is discharged, via the discharge circuit, through the patient.

In further enhancements, a Bluetooth Smart or equivalent comlink could be created between the either the portable antenna or the charger antenna and a Home Automation system (i.e. Smart Home). Connection to a Home Automation system could allow the WCD System to access and control overhead lighting, house speakers and other systems within the patient's home, further enriching communication to the patient and their family members.

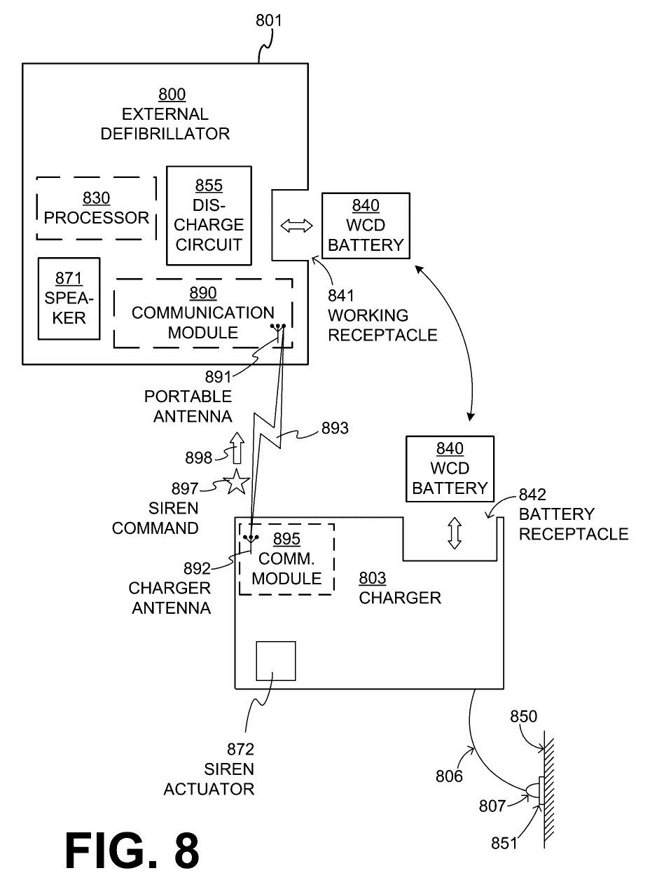

FIG. 8 is a diagram of sample components of a WCD system that is made according to embodiments. The WCD system includes an external defibrillator 800 that is provided in a housing 801. External defibrillator 800 can be as external defibrillator 200 or 100, and can be coupled to a support structure that is not shown in FIG. 8, and could be as support structure 170. The WCD system moreover includes components that are coupled to a support structure such as support structure 170. In the example of FIG. 8, many of these components are part of external defibrillator 800, as was done in FIG. 3. More particularly, external defibrillator 800 includes a discharge circuit 855 that can be as discharge circuit 255, optionally a communication module 890 that can be as communication module 290, a portable antenna 891 that can be as portable antenna 291, and optionally a processor 830 that can be as processor 230. Moreover, the components of the WCD system of FIG. 8 may further include components described with reference to FIG. 3, a communicator receptacle as is described later, and so on.

The WCD system also includes a rechargeable WCD battery 840, which can be inserted in a working receptacle 841 for powering external defibrillator 800. WCD battery 840 can also be removed from working receptacle 841, for being recharged by a charger 803 that is made according to embodiments.

Charger 803 can be as charger 103. Charger 803 can be configured to be coupled to an electrical wall outlet 851 of a wall 850, so as to receive electrical power. More particularly, charger 803 may have an electrical cord 806 with a plug 807 that can be plugged into electrical wall outlet 851.

Charger 803 can be configured to charge WCD battery 840 from the electrical power received via electrical cord 806, while WCD battery 840 is uncoupled from external defibrillator 800 and is thus also uncoupled from discharge circuit 855. This charging process, therefore, causes WCD battery 840 to store an electrical charge, which can be used as mentioned above. Preferably, charger 803 includes a battery receptacle 842 that is configured to receive WCD battery 840 while charger 803 charges WCD battery 840.

Charger 803 may further include a siren actuator 872. Siren actuator 872 may be a part of a broader user interface of charger 803. Siren actuator 872 may be implemented as a button, a switch, a touchscreen that can receive a user input, and so on. Siren actuator 872 can be actuated by a human attendant other than the patient, for uses described later. For actuating, siren actuator 872 can be configured to receive a siren input by the human attendant.

Charger 803 may also include a charger antenna 892. In some embodiments, charger 803 has its own communication module 895 that is powered by the electrical power, and charger antenna 892 can be part of communication module 895. Charger antenna 892 can be configured to transmit wirelessly a siren command 897 responsive to the siren input received by siren actuator 872. Siren command 897 may travel via a comlink 893 along a direction 898, and be thus received by portable antenna 891, which is configured to receive it. Comlink 893 may be implemented as comlink 393.

The WCD system further includes a speaker 871. Speaker 871 can be coupled to the support structure of the WCD system. In the example of FIG. 8, speaker 871 is part of external defibrillator 800. Speaker 871 may be used in a number of ways.

In some embodiments, speaker 871 is configured to emit a sound responsive to portable antenna 891 receiving siren command 897. In some embodiments, the sound is intended to communicate a message to the patient. This can be accomplished by having the sound have an intensity of about 55 dB. A plurality of messages can be effectuated, for example by being encoded in the siren command, and so on.

In some embodiments, processor 830 is configured to determine that the stored electrical charge should be discharged through the patient, similarly with embodiments of processor 330. Speaker 871 can be configured to emit a sound responsive to the determination, for example as described above.

In other embodiments, the attendant uses the siren command to locate the patient, when the patient is not responding. This can be, for example, when charger 803 further includes a user interface as described above for user interface 371, processor 830 has determined that the stored electrical charge should be discharged through the patient, an encoded message has been sent that encodes the determination, and a human-perceptible indication has been made by speaker 871 prior to the discharge, and so on. At that time, the attendant may want to find the patient, and the sound is intended to operate as a siren for the attendant to find the patient.

In some embodiments, the attendant is able to progressively increase the intensity of the sound, until he or she can hear it. For example, the siren actuator can be configured to receive a subsequent siren input that is of the same or different type than the previous siren input. The charger antenna can be configured to transmit wirelessly a subsequent siren command responsive to the received subsequent siren input, and the speaker can be configured to emit a subsequent sound responsive to the portable antenna receiving the subsequent siren command. The subsequent sound may have an intensity larger by at least 5 dB than the original sound. Subsequent sounds may have even higher intensity, and so on.

Additional features may be implemented. For example the portable portion of the system may have an option to silence speaker 871, so as to not let it become intolerable in case the patient is fine and the attendant is misinformed or is making a mistake.

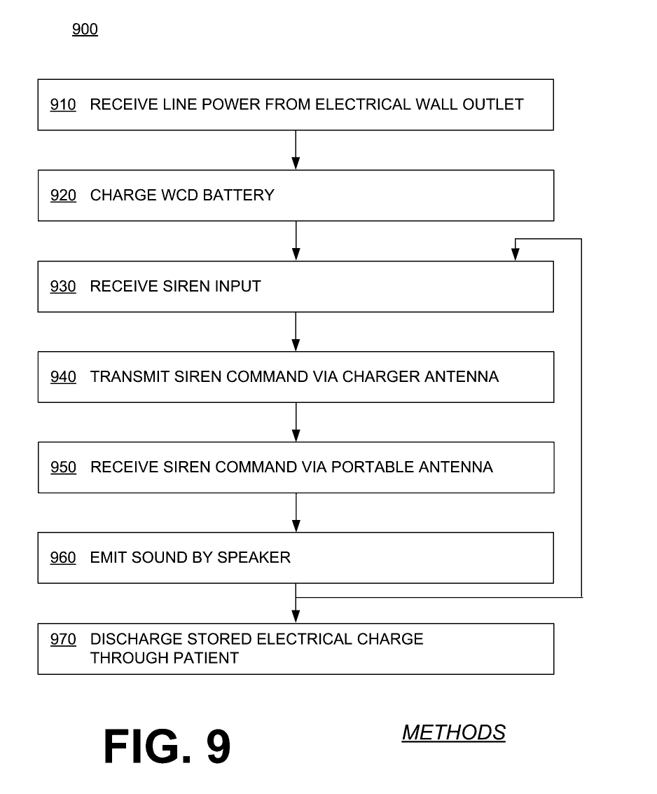

FIG. 9 shows a flowchart 900 for describing methods according to embodiments. The methods of flowchart 900 may be practiced by embodiments described above.

According to an operation 910, electrical power is received in the charger. The electrical power may be received from an electrical wall outlet.

According to another operation 920, a WCD battery may be charged with the electrical power received at operation 910. This charging will result in the WCD battery to store an electrical charge. In some embodiments, the charger further includes a communicator receptacle configured to receive a mobile communication device that has a suitable rechargeable battery that is also known as a communicator battery. In such embodiments, the communicator battery can be further charged from the electrical power, while the mobile communication device has been received in the communicator receptacle.

According to another operation 930, a siren input can be received via the siren actuator. The siren input may have been entered by a human attendant other than the patient.

According to another operation 940, a siren command can be transmitted wirelessly via the charger antenna. The siren command can be transmitted responsive to the received siren input.

According to another operation 950, the siren command can be received via the portable antenna.

According to another operation 960, a sound is emitted by the speaker, responsive to receiving the siren command. Operations 930, 940, 950 and 960 may be repeated subsequent times, sometimes to progressively increase the intensity of the sound.

According to another operation 970, the stored electrical charge is discharged, via the discharge circuit, through the patient. In some embodiments, it is determined that the stored electrical charge should be discharged through the patient and, in such embodiments, a sound can be emitted by the speaker responsive to the determination.

In the methods described above, each operation can be performed as an affirmative step of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

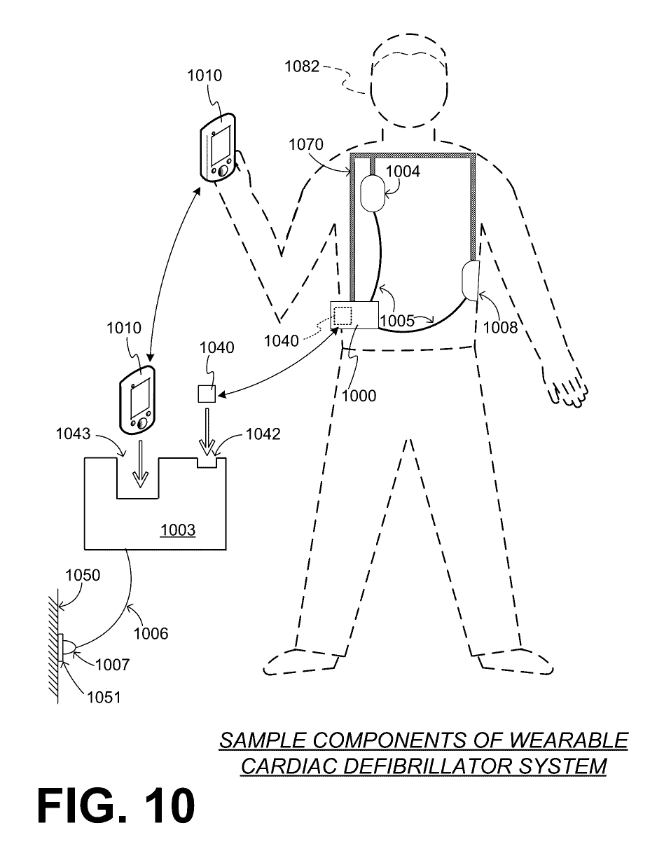

FIG. 10 is a diagram of components of a sample wearable cardiac defibrillator (WCD) system, made according to embodiments. The WCD system of FIG. 10 is configured for use by a patient 1082 who may communicate via a mobile communication device 1010. Mobile communication device 1010 has a communicator battery (not shown). Mobile communication device 1010 can be part of the WCD system, or be a regular, commercially available mobile communication device, with or without an application that is related to the WCD system.

The WCD system of FIG. 10 includes a support structure 1070 that is configured to be worn by patient 1082, similarly with support structure 170. It also includes an external defibrillator 1000 that is coupled to support structure 1070, and electrodes 1004, 1008 that are coupled to external defibrillator 1000 via electrode leads 1005.

The WCD system of FIG. 10 includes a discharge circuit that is not shown, which in this example is within external defibrillator 1000. The discharge circuit is thus coupled to support structure 1070. The discharge circuit is configured to discharge through patient 1082 electrical charge that is stored in WCD battery 1040, an energy storage module, and so on as per the above.

The WCD system of FIG. 10 includes a charger 1003. Charger 1003 can be configured to be coupled to an electrical wall outlet 1051 of a wall 1050, so as to receive electrical power. More particularly, charger 1003 may have an electrical cord 1006 with a plug 1007 that can be plugged into electrical wall outlet 1051.

Charger 1003 has a battery receptacle 1042 and a communicator receptacle 1043 that has a different shape than battery receptacle 1042. Because there are two dissimilar chargers, charger 1003 can also be called a combination charger.

The WCD system of FIG. 10 additionally includes a rechargeable WCD battery 1040, which is configured to store an electrical charge. In FIG. 10, WCD battery 1040 is shown in two places, both completely inserted in external defibrillator 1000, and also being placed in battery receptacle 1042 for being recharged. WCD battery 1040 is configured to be electrically coupled to the discharge circuit when inserted in external defibrillator 1000, and also uncoupled from the discharge circuit for being recharged. For recharging, battery receptacle 1042 can be configured to receive WCD battery 1040 while the latter is uncoupled from the discharge circuit. Plus, charger 1003 can be configured to further charge WCD battery 1040 from the electrical power while WCD battery 1040 is received in battery receptacle 1042. Thus WCD battery 1040 stores electrical charge that can be discharged through the discharge circuit.

Communicator receptacle 1043 is configured to receive mobile communication device 1010. Charger 1003 can be further configured to charge the communicator battery from the electrical power while mobile communication device 1010 is received in communicator receptacle 1043. Other features described above may also be implemented in the system of FIG. 10. For example, charger 1003 may have a user interface such as user interface 371, a siren actuator such as siren actuator 872, a charger antenna, and so on.

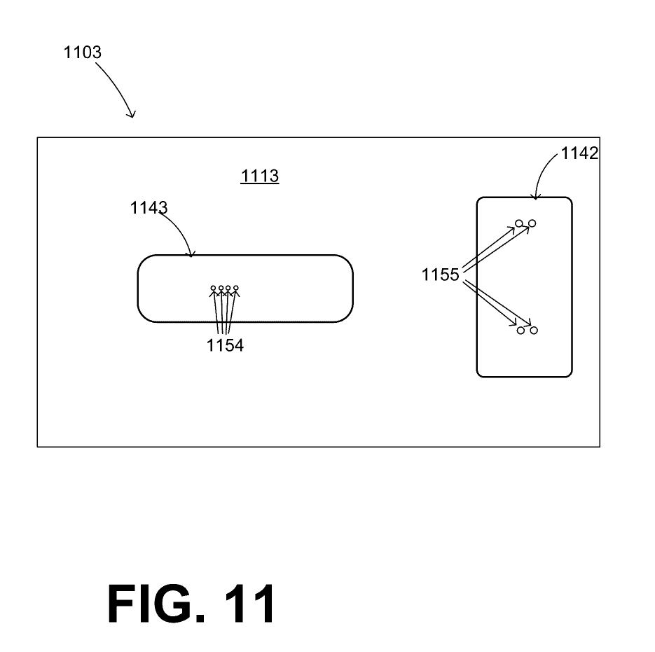

FIG. 11 is a top view of a combination charger 1103 of a WCD system, which is made according to embodiments. Combination charger 1103 could be charger 1003.

Combination charger 1103 has a top surface 1113. Combination charger 1103 also has a battery receptacle 1142 and a communicator receptacle 1143 that are well-like openings in top surface 1113. Battery receptacle 1142 has a different shape than communicator receptacle 1143 in this example.

In the example of FIG. 11, charging is conductive. Battery receptacle 1142 has charging and/or data pins 1155, while communicator receptacle 1143 has charging and/or data pins 1154. In each instance, the pins are not placed in a symmetric position with respect to each of well so as to guide placement with at correct orientation, although this is not required can be avoided with planning the pin functions, etc.

A person skilled in the art will be able to practice the present invention in view of this description, which is to be taken as a whole. Details have been included to provide a thorough understanding. In other instances, well-known aspects have not been described, in order to not obscure unnecessarily this description. Plus, any reference to any prior art in this description is not, and should not be taken as, an acknowledgement or any form of suggestion that such prior art forms parts of the common general knowledge in any country or any art.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In this document, the phrases “constructed to” and/or “configured to” denote one or more actual states of construction and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Any and all parent, grandparent, great-grandparent, etc. patent applications, whether mentioned in this document or in an Application Data Sheet (ADS) of this patent application, are hereby incorporated by reference herein, including any priority claims made in those applications and any material incorporated by reference, to the extent such subject matter is not inconsistent herewith.

In this description a single reference numeral may be used consistently to denote a single item, aspect, component, or process. Moreover, a further effort may have been made in the drafting of this description to choose similar though not identical reference numerals to denote versions or embodiments of an item, aspect, component or process that possibly similar or different. Where made, such a further effort was not required, but was nevertheless made gratuitously to accelerate comprehension by the reader. Even where made in this document, such an effort might not have been made completely consistently for all of the versions or embodiments that are made possible by this description. Accordingly, the description controls in defining the item, aspect, component or process, rather than its reference numeral. Any similarity in reference numerals may be used to infer a similarity in the text, but not to confuse aspects where the text or the context indicates otherwise.

The claims of this document define certain combinations and subcombinations of elements, features and steps or operations, which are regarded as novel and non-obvious. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that it can have one or more of this component or item.

1. A wearable cardiac defibrillator (WCD) system, comprising:
a support structure configured to be worn by a patient;
a discharge circuit coupled to the support structure;
a monitoring device coupled to the support structure and configured to monitor a physiological parameter of the patient;
a portable antenna coupled to the support structure, the portable antenna configured to transmit wirelessly an encoded message from the monitoring device;
a WCD battery configured to store an electrical charge, the WCD battery further configured to be electrically coupled to and uncoupled from the discharge circuit, the discharge circuit configured to discharge the stored electrical charge through the patient; and
a charger configured to be coupled to an electrical wall outlet so as to receive electrical power, the charger further configured to charge the WCD battery from the received electrical power while the WCD battery is uncoupled from the discharge circuit, so that the WCD battery stores the electrical charge, the charger including:
a charger antenna configured to receive the encoded message, and
a user interface configured to output a human-perceptible indication responsive to the charger antenna receiving the encoded message, in which the human-perceptible indication includes a message about a value of the monitored physiological parameter.

2. The WCD system of claim 1, in which
the charger further includes a battery receptacle configured to receive the WCD battery while the charger charges the WCD battery.

3. The WCD system of claim 1, further comprising:
an energy storage module configured to receive the electrical charge stored in the WCD battery, and to store the received electrical charge, and
in which the discharge circuit is configured to discharge the electrical charge that becomes stored in the energy storage module.

4. The WCD system of claim 1, in which
the charger further includes a memory configured to store a data group,
the stored data group is sent to the user interface responsive to the charger antenna receiving the encoded message, and
the human-perceptible indication is output by the user interface responsive to the data group being sent to the user interface.

5. The WCD system of claim 1, in which
the charger further includes a memory configured to store a plurality of data groups,
a message code is decoded from the encoded message received by the charger antenna,
at least a certain one of the data groups is selected responsive to the message code,
the certain data group is sent to the user interface, and
the human-perceptible indication is output by the user interface responsive to the certain data group being sent to the user interface.

6. The WCD system of claim 1, in which
the human-perceptible indication includes a message about a system parameter of the WCD system.

7. (canceled)

8. The WCD system of claim 1, further comprising:
a processor configured to determine that the stored electrical charge should be discharged through the patient, and
in which the human-perceptible indication includes a message about the determination.

9. The WCD system of claim 1, further comprising:
a processor configured to determine that the stored electrical charge should be discharged through the patient at a certain moment unless there is a timely intervention by a human, and
in which the human-perceptible indication includes a message about the determination, and
the encoded message is transmitted at least 4 sec prior to the certain moment.

10. The WCD system of claim 1, in which
the human-perceptible indication includes a sound, and
the user interface includes a speaker configured to play the sound.

11. The WCD system of claim 1, in which
the human-perceptible indication includes a visual message, and
the user interface includes a screen configured to display the visual message.

12. The WCD system of claim 1, in which
the human-perceptible indication includes a visual message, and
the user interface includes a panel with a portion that displays the visual message, and a light source associated with the panel portion.

13. The WCD system of claim 1, further comprising:
a speaker coupled to the support structure; and
in which the charger further includes a siren actuator configured to receive a siren input by a human attendant distinct from the patient,
the charger antenna is configured to transmit wirelessly a siren command responsive to the received siren input,
the portable antenna is configured to receive the siren command, and
the speaker is configured to emit a sound responsive to the portable antenna receiving the siren command.

14. The WCD system of claim 1, in which
the charger further includes a battery receptacle configured to receive the WCD battery while the charger charges the WCD battery,
the charger further includes a communicator receptacle configured to receive a mobile communication device that has a communicator battery, the communicator receptacle made differently than the battery receptacle, and
the charger is further configured to charge the communicator battery from the electrical power while the mobile communication device has been received in the communicator receptacle.

15. A method for a wearable cardiac defibrillator (WCD) system to be used by a patient, the WCD system including a WCD battery, a support structure configured to be worn by the patient, a discharge circuit coupled to the support structure, a monitoring device coupled to the support structure and a portable antenna coupled to the support structure, the WCD system further including a charger configured to be coupled to an electrical wall outlet, the charger including a charger antenna and a user interface, the method comprising:
receiving, in the charger, electrical power from the electrical wall outlet;
charging, via the charger, the WCD battery with the received electrical power so that the WCD battery stores an electrical charge;
monitoring a physiological parameter of the patient by the monitoring device;
transmitting, via the portable antenna, wirelessly an encoded message from the monitoring device;
receiving, via the portable antenna the encoded message;
outputting, via the user interface, a human-perceptible indication responsive to the charger antenna receiving the encoded message, in which the human-perceptible indication includes a message about a value of the monitored physiological parameter; and
discharging, via the discharge circuit, the stored electrical charge through the patient.

16. The method of claim 15, in which
the charger further includes a memory, and
further comprising:
storing a data group in the memory; and
sending the stored data group to the user interface responsive to the charger antenna receiving the encoded message, and
in which the human-perceptible indication is output by the user interface responsive to the data group being sent to the user interface.

17. The method of claim 15, in which
the charger further includes a memory, and
further comprising:
storing a plurality of data groups in the memory;
decoding a message code from the received encoded message;
selecting at least a certain one of the data groups responsive to the message code;
sending the certain data group to the user interface, and
in which the human-perceptible indication is output by the user interface responsive to the certain data group being sent to the user interface.

18. The method of claim 15, in which
the human-perceptible indication includes a message about a system parameter of the WCD system.

19. (canceled)

20. The method of claim 15, further comprising:
determining that the stored electrical charge should be discharged through the patient, and
in which the human-perceptible indication includes a message about the determination.

21. The method of claim 15, in which
the charger further includes a battery receptacle configured to receive the WCD battery while the charger charges the WCD battery,
the charger further includes a communicator receptacle configured to receive a mobile communication device that has a communicator battery, the communicator receptacle made differently than the battery receptacle, and
further comprising:
charging the communicator battery from the electrical power while the mobile communication device has been received in the communicator receptacle.

22-34. (canceled)
